(19) United States
(12) Patent Application Publication
(10) Pub. No.: US 2026/0002929 A1
(43) Pub. Date: Jan. 1, 2026

(54) DIAGNOSTIC ENGINEERED IMMUNE SKEWING MODEL FOR PREDICTIVE CANCER METASTASIS

(71) Applicant: The Trustees of Columbia University in the City of New York, New York, NY (US)

(72) Inventors: Daniel Naveed TAVAKOL, New York, NY (US); Gordana VUNJAK-NOVAKOVIC, New York, NY (US); Pamela L. GRANEY, New York, NY (US)

(21) Appl. No.: 19/320,055

(22) Filed: Sep. 5, 2025

Related U.S. Application Data
(63) Continuation of application No. PCT/US2024/019642, filed on Mar. 13, 2024.
(60) Provisional application No. 63/490,976, filed on Mar. 17, 2023.

(51) Int. Cl. G01N 33/50; A61B 10/00; C12N 5/09

(52) U.S. Cl. CPC G01N 33/5011; C12N 5/0693; G01N 33/5082; A61B 10/0041; C12N 2503/04; C12N 2513/00; C12N 2533/90

(57) ABSTRACT

Models and methods are provided for non-invasive diagnostic and predictive assessment of the systemic effects of metastasis in a human based on a tissue engineered immune system. Characterization of changes to bone marrow resulting from the presence and progression of a primary breast tumor including skewing of blood/immune cells at various phenotypes of cancer progression, and its use in assessing the metastatic state of a subject and/or the effects of candidate drugs thereon.

### CROSS-REFERENCE TO RELATED APPLICATIONS

This application is a continuation of PCT International Application No. PCT/US2024/019642, filed Mar. 13, 2024, which claims benefit of U.S. Provisional Application No. 63/490,976, filed Mar. 17, 2023, the contents of each of which are hereby incorporated by reference.

### GOVERNMENT SUPPORT CLAUSE

This invention was made with government support under contract no. CA249799 and CA275733 and EB027062 awarded by the National Institutes of Health. The government has certain rights in the invention.

### INTRODUCTION

Cancer continues to claim millions of lives each year. Cancer metastasis, responsible for 90% of cancer-related mortalities, remains difficult to treat, leaving an ongoing tumor burdens for patients with metastatic cancers even if in remission. Current cancer therapeutic approaches are based on either killing all rapidly dividing cells or targeting a specific tumor mutation, and neither approach is sufficiently effective if the patient has metastasis.

Decades of biological research in cell culture and animal models have tremendously advanced our understanding of cancer. However, these models recapitulate human cancers to only limited extent (only about 5% of cancer therapeutics that passed screening show efficacy in clinical trials) and fail to recapitulate metastatic disease. In addition, cancer cells and their metastatic potential and target organs are highly heterogeneous, both among the patients and within a single tumor. Together, these factors have contributed to the current lack of treatment options. While mice (i) reproduce and mature quickly, (ii) can be genetically defined, (iii) contain biological complexity, (iv) present a neoplastic development similar to that in humans, and (v) support the growth of implanted human cancer cells, they fail to mimic key aspects of human physiology. Notably, mice lack human immune components, fail to display metastasis to expected organ sites, and differ from humans in their genetic drift and clonal expansion of tumor cells.

The BM is a major regulator of homeostasis and disease, as the residing site of HSPCs. The functional role of the BM is to produce downstream blood and immune cells, acting as a renewable source of myeloid and lymphoid progenitors that can respond to systemic stimuli in injury, regeneration and disease. In the presence of a primary tumor, circulating tumor-secreted factors travel to the BM and redirect the lineage commitment of HSPCs, causing accumulation of myeloid-derived suppressor cells (MDSCs) that promote tumor progression. It is still poorly understood how the systemic perturbations to the BM, distinct from those occurring at the primary tumor site, alter the BM niche and how these changes bias hematopoiesis toward myeloid-skewing, limiting our ability to develop effective immunotherapies.

There is a huge need for human-specific tissue models that would more accurately predict the progression of cancer and responses to treatment, ideally in a patient-specific context. Beyond genetically engineered mouse models, which spontaneously form cancers, researchers have developed PdX mouse models by injecting patient's tumor cells into immunodeficient mice. While PdX models offer patient-specific insights, they also require significant time and effort to establish, do not display the immune system of the patient, and have been shown to induce rapid mutational drifting in the transplanted cells. More recently, human tissue models of cancer, in form of organoids and organ-on-chips, have emerged as patient specific mimics for studies of cancer progression. Notably, these models can be tailored to capture the individual aspects of cancer, while allowing a range of complexity, depending on the question asked. This feature is particularly valuable for studies of late-stage and metastatic cancers, where currently available models fail to capture cancer progression and response (or resistance) to treatment. A number of human tissue models have been developed to mimic various types and stages of cancer, from primary tumors and their niches, to intravasation and extravasation of circulating tumor cells, systemic crosstalk, metastatic preconditioning, and metastasis.

### SUMMARY

A 2D or 3D in vitro model of a human bone marrow state comprising human hematopoietic stem and progenitor cells (HSPC) and conditioned culture media comprising (i) media obtained from a primary human triple-negative metastatic breast cancer (TNBC) culture, or (ii) media obtained from a human TNBC cell line, or (iii) plasma or serum obtained from a human subject with TNBC.

A 2D or 3D in vitro model of a human bone marrow state comprising human hematopoietic stem and progenitor cells (HSPC) and conditioned culture media comprising (i) media obtained from a culture of human hormone receptor (HR)+ culture or serum, or plasma from a sufferer thereof, (ii) media obtained from a culture of Luminal A, Luminal B, HER2+ or basal-like (triple negative) culture, or serum or plasma from a sufferer thereof.

A method of determining if a candidate drug has an ameliorative effect on immune skewing in a bone marrow associated with a metastatic cancer comprising contacting a 2D or 3D in vitro model as described herein with the candidate drug and quantifying the amount of immune skewing associated cells in the 2D or 3D in vitro model before and after contacting with the candidate drug so as to thereby determine if the candidate drug reduces or not the amount of immune skewing associated cells in the 2D or 3D in vitro model, wherein a reduction in the amount of immune skewing associated cells in the 2D or 3D in vitro model after contact with the candidate drug indicates that the candidate drug has an ameliorative effect on immune skewing in a bone marrow associated with a metastatic cancer, and wherein no reduction in the amount of immune skewing associated cells in the 2D or 3D in vitro model after contact with the candidate drug indicates that the candidate drug does not have an ameliorative effect on immune skewing in a bone marrow associated with a metastatic cancer.

A method of determining a metastatic state or metastatic potential of a breast cancer tumor in a subject, comprising contacting a 2D or 3D in vitro model as described herein wherein said conditioned culture media, comprising serum or plasma, comprises serum or plasma obtained from said subject, and determining the resultant amount and identity of immune skewing associated cells in the 2D or 3D in vitro model, and correlating the amount and identity of immune skewing associated cells with predetermined control indicating the metastatic state or metastatic potential of a breast cancer tumor in the subject.

### BRIEF DESCRIPTION OF THE DRAWINGS

FIGS. 1A-1B: Overview of biological phenomenon of cancer metastasis. (1A) Altering the hematopoietic niche; (1B) envisioned experimental goals for using serum to understand changes to hematopoiesis and myeloid cell production in eBM tissues.

FIGS. 2A-2B: 2D culture of CB-HSPCs with TNBC-derived conditioned media induces myeloid skewing. (2A) Experimental overview, including cell lines used and media compositions. (2B) Example flow cytometry gating of CD11b+CD14+ myeloid cells in response to treatment with TNBC-derived conditioned media.

FIGS. 3A-3B: Flow cytometry reveals changes in myeloid skewing of HSPCs after exposure to TNBC-derived conditioned media with and without serum. (3A) Changes to myeloid progeny from HSPCs in conditioned media containing serum. (3B) Changes to myeloid progeny from HSPCs in conditioned media containing no-serum (serum-starved TNBC cell lines). *p<0.05; ** p<0.01; *** p<0.005; **** p<0.001 with One-Way ANOVAs with Tukey's post-hoc analysis.

FIG. 4: Cytokine analysis of cultured medium from HSPCs reveals increased inflammatory factors after exposure to TNBC-derived conditioned media in 2D. One-Way ANOVAs with Tukey's post-hoc analysis. ‘a’ p<0.05 relative to all other groups; ‘b’ p<0.05 relative to all other groups; ‘#’ p<0.05 relative to MDA, LM2, and BoM.

FIG. 5: Experimental design in TNBC conditioned media exposure to 3D eBM tissues. Metastatic breast cancer conditioned medium induces myeloid skewing of HSPCs in 3D eBMs.

FIGS. 6A-6B: Flow cytometry reveals unique changes to MDSCs over 4 and 10 days in 3D eBM microtissues. Changes in myeloid cells at D4 (A) and Day 10 (B), with conditioned from basal media “C”, MCF10 cells “F”, lung-“L” and bone-“B” tropic cancer cells, and MDA-MB-231 cells “M”. * p<0.05; ** p<0.01; *** p<0.005; **** p<0.001 with One-Way ANOVAs with Tukey's post-hoc analysis.

FIGS. 7A-7C. Dose-dependent response of patient serum & plasma in 2D HSPCs. Flow cytometry reveals a dose-dependent response of HSPCs to breast cancer serum in (A) CD11b+CD14+ monocytes, (B) CD11b+CD14+CD11c+ dendritic cells, and (C) CD11b+CD14+CD15+ MDSCs. * p<0.05; ** p<0.01; with One-Way ANOVAs.

FIGS. 8A-8B: Myeloid-skewing phenotype is exhibited in 2D HSPCs and 3D eBM microtissues after exposure to different stages of breast cancer serum. Flow cytometry reveals changes to myeloid progeny at Days 4 and 8 in 2D HSPCs in monolayer (8A) as well as at Day 8 in 3D eBM microtissues (8B). * p<0.05; ** p<0.01; *** p<0.005; **** p<0.001 with One-Way ANOVAs.

### DETAILED DESCRIPTION

A novel non-invasive diagnostic and predictive tool for assessing the systemic effects of metastasis in a human, tissue engineered immune system. Notably, this system enables characterization of changes to the BM resulting from the presence and progression of a primary breast tumor (in the absence of tumor cells themselves, just their secreted factors), including skewing of blood/immune cells at various phenotypes of cancer progression.

A 2D or 3D in vitro model of a human bone marrow state comprising human hematopoietic stem and progenitor cells (HSPC) and conditioned culture media comprising (i) media obtained from a primary human triple-negative metastatic breast cancer (TNBC) culture, or (ii) media obtained from a human TNBC cell line, or (iii) plasma or serum obtained from a human subject with TNBC. HSPC are known in the art and are a population of precursor cells that possess the capacity for self-renewal and multilineage differentiation, and can be found in BM. They can also be isolated from mobilized peripheral blood or umbilical cord blood.

TNBC are well known in the art and are breast cancers which do not have estrogen or progesterone receptors (ER or PR) and also do not make any, or only low amounts of, HER2.

A 2D or 3D in vitro model of a human bone marrow state comprising human hematopoietic stem and progenitor cells (HSPC) and conditioned culture media comprising (i) media obtained from a culture of human hormone receptor (HR)+ culture or serum, or plasma from a sufferer thereof, (ii) media obtained from a culture of Luminal A, Luminal B, HER2+ or basal-like (triple negative) culture, or serum or plasma from a sufferer thereof.

In embodiments, the HSPC have previously been obtained from a human. In embodiments, the HSPCs are obtained from bone marrow, mobilized peripheral blood or umbilical cord blood.

In embodiments, the 2D or 3D in vitro model is a 3D model and which comprises a decellularized bone scaffold. In embodiments, the decellularized bone scaffold is non-human mammalian. In embodiments, the decellularized bone scaffold comprises bone from a human.

In embodiments the model is a 2D model. In embodiments, the model is present in a chip or in a well of a multiwell device.

In embodiments, the TNBC cell line comprises a MDA-MB-231 breast cancer cell line, lung-targeting (LM2), and/or bone-targeting (BOM) cell line. MDA-MB-231 is widely available, including from the ATCC. LM2 are also known in the art, see, e.g., Nature. 2005 Jul. 28; 436 (7050): 518-524 (doi: 10.1038/nature03799). BOM are also known in the art, e.g., see MDA-231-BOM-1833: Human Breast Adenocarcinoma Cell Line available from MSKCC or MD Anderson.

In embodiments, the conditioned culture media has been supplemented with fetal bovine serum.

In embodiments, the conditioned culture media is not supplemented with fetal bovine serum.

In embodiments, the conditioned culture media comprises plasma or serum obtained from a human subject with TNBC.

In embodiments, the conditioned culture media comprises about 1%-2% plasma or serum obtained from a human subject with TNBC.

In embodiments, the conditioned culture media comprises greater than 0.5% plasma or serum obtained from a human subject with TNBC.

Culture media of the models can also contain conventional basal media, such as Dulbecco's Modified Eagle Medium (DMEM).

In embodiments, the model comprises a greater number of total myeloid cells relative to an otherwise identical 2D or 3D in vitro model cultured with basal media and no conditioned culture media comprising media obtained from (i), (ii) or (iii).

In embodiments, the model comprises a greater number of CD11b+CD14+CD15+ myeloid-derived suppressor cells (MDSCs) relative to an otherwise identical 2D or 3D in vitro model cultured with basal media and no conditioned culture media comprising media obtained from (i), (ii) or (iii).

In embodiments, the or serum or plasma is from a subject having a Stage I, II, III or IV cancer.

A method of determining if a candidate drug has an ameliorative effect on immune skewing in a bone marrow associated with a metastatic cancer comprising contacting a 2D or 3D in vitro model as described herein with the candidate drug and quantifying the amount of immune skewing associated cells in the 2D or 3D in vitro model before and after contacting with the candidate drug so as to thereby determine if the candidate drug reduces or not the amount of immune skewing associated cells in the 2D or 3D in vitro model, wherein a reduction in the amount of immune skewing associated cells in the 2D or 3D in vitro model after contact with the candidate drug indicates that the candidate drug has an ameliorative effect on immune skewing in a bone marrow associated with a metastatic cancer, and wherein no reduction in the amount of immune skewing associated cells in the 2D or 3D in vitro model after contact with the candidate drug indicates that the candidate drug does not have an ameliorative effect on immune skewing in a bone marrow associated with a metastatic cancer.

In embodiments, the candidate drug is a small molecule.

In embodiments, the candidate drug is a biologic.

In embodiments, the candidate drug is a CAR-T.

A method of determining a metastatic state or metastatic potential of a breast cancer tumor in a subject, comprising contacting a 2D or 3D in vitro model as described herein wherein said conditioned culture media, comprising serum or plasma, comprises serum or plasma obtained from said subject, and determining the resultant amount and identity of immune skewing associated cells in the 2D or 3D in vitro model, and correlating the amount and identity of immune skewing associated cells with predetermined control indicating the metastatic state or metastatic potential of a breast cancer tumor in the subject.

In embodiments, the method further comprises obtaining the serum or plasma from said subject.

In embodiments, the serum is used.

In embodiments, CD11b+CD14+CD15+ myeloid-derived suppressor cells are identified and/or quantified and, optionally, compared to a predetermined control.

In embodiments, the total myeloid cells are identified and/or quantified and, optionally, compared to a predetermined control.

In embodiments, the immune skewing associated cells are identified and/or quantified with flow cytometry.

In embodiments, the method further comprises performing a cytokine analysis on immune skewing associated cells of the model. In embodiments, the cytokine analysis comprises measuring IFN-α2, IFN-g, TNF-α, MCP-1, and/or IL-8, among others.

In embodiments, the method further comprises measuring IL-10 levels.

In embodiment the predetermined controls are determined from samples from subjects with a diagnosed staged cancer or diagnosed metastatic state.

In some embodiments, a three-dimensional decellularized bone scaffold is provided for an eBM model, and includes a plurality of cells arrayed on the scaffold. In some embodiments, the bone scaffold comprises a plurality of perfusion channels. In some embodiments, the plurality of cells comprises patient-derived cells. In some embodiments, the scaffold is adapted for insertion in one well of a multiple well plate. In some embodiments, the scaffold is adapted for insertion in one well of a 96-well plate. In some embodiments, the scaffold is adapted for insertion in one well of a 24-well plate. In some embodiments, the scaffold has an outer region, and inner region, and a core region. In embodiments, hematopoietic components are seeded onto the scaffold with fibrinogen. In embodiments, hematopoietic components are expanded prior to seeding.

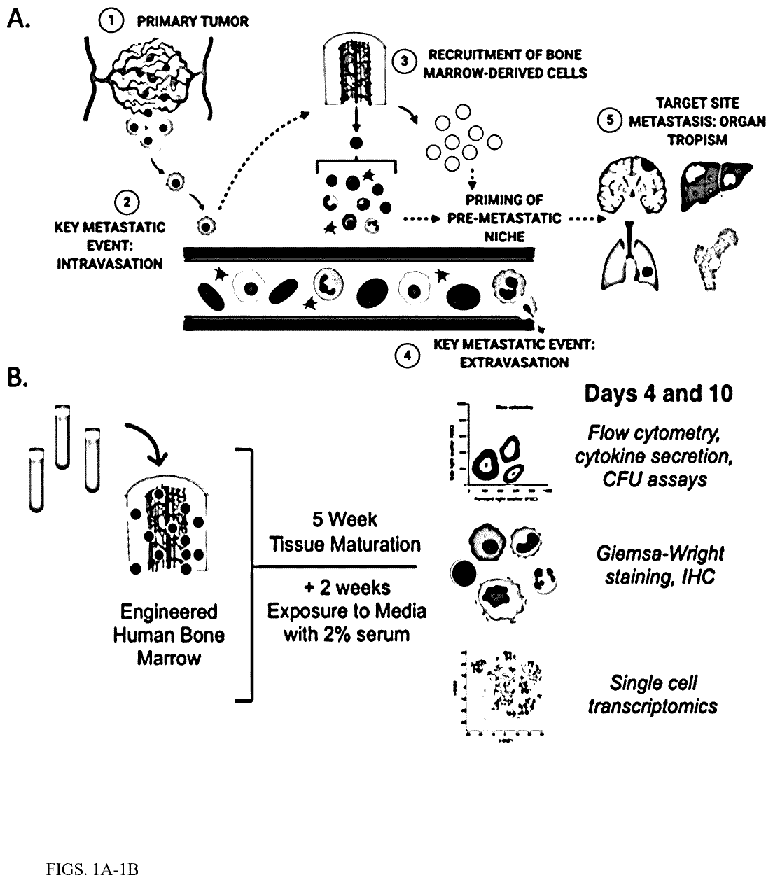

Our group has recently established an engineered model of the healthy bone marrow (eBM), producing downstream blood/immune cells in response to external perturbations, such as injury and bio/chemical factors, such as those in patient's serum and plasma. Notably, this model enables us to study the crosstalk between the niche and HSPCs during hematopoiesis in both health and disease. Here we describe use of our eBM model to characterize the response to tumor-released circulating signals acting on the BM, to reshape the immune cell landscape in the BM and promote metastasis (FIGS. 1A-1B).

In this work, we investigated how systemic effects of metastasis on eBM prior, during, and after metastatic colonization of bone by breast cancer cells. As altered hematopoiesis is a hallmark of metastasis and is patient-specific, we assessed the changes in the BM during breast cancer that may drive tumor progression and metastasis, using the individualized eBM models. A healthy engineered 3D microenvironment capable of supporting HSPCs in vitro can be used to recapitulate the tumor-induced changes to hematopoiesis and the stromal niche that occur in breast cancer patients. We describe here the use of our eBM model for studying changes in metastatic cancer cell lines from triple-negative metastatic breast cancer patients (TNBC), as well as applications of these methodologies for serum from patients with hormone receptor (HR)+ and TNBC+ metastatic tumors. This study forms a scientific premise and technological basis for delineating the contributions of cancer stage and subtype on malignant cell differentiation, as a predictive tool during the stages of malignancy before metastatic cancer cells have physically reached the BM.

### Results

Metastatic breast cancer conditioned medium induces myeloid skewing of HSPCs in monolayer: To investigate tumor-induced hematopoietic skewing, we used culture media supernatant from TNBC cell lines to demonstrate a bias toward myeloid lineages of HSPCs in 2D suspension culture (FG. 2A). HSPCs were cultured in hematopoietic basal media (SFEM II, Stem Cell Tech) supplemented with conditioned media from a parent MDA-MB-231 breast cancer cell line, lung-targeting (LM2) and bone-targeting (BOM) subsets (e.g., as established by the Joan Massagué lab at MSKCC), a control breast epithelial line (MCF10A), and healthy basal media control. Conditioned medium was collected from each cell line with and without the addition of 10% FBS, which is normally supplemented in MDA-MB-231, LM2, and BOM cells and absent in MCF10A cells.

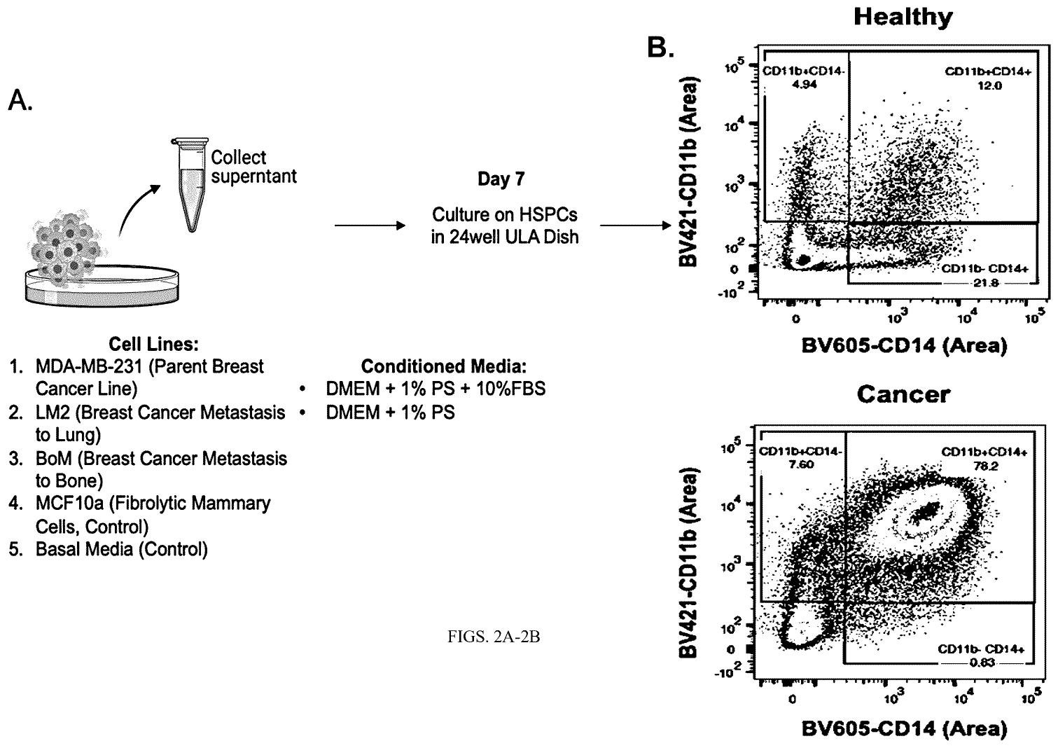

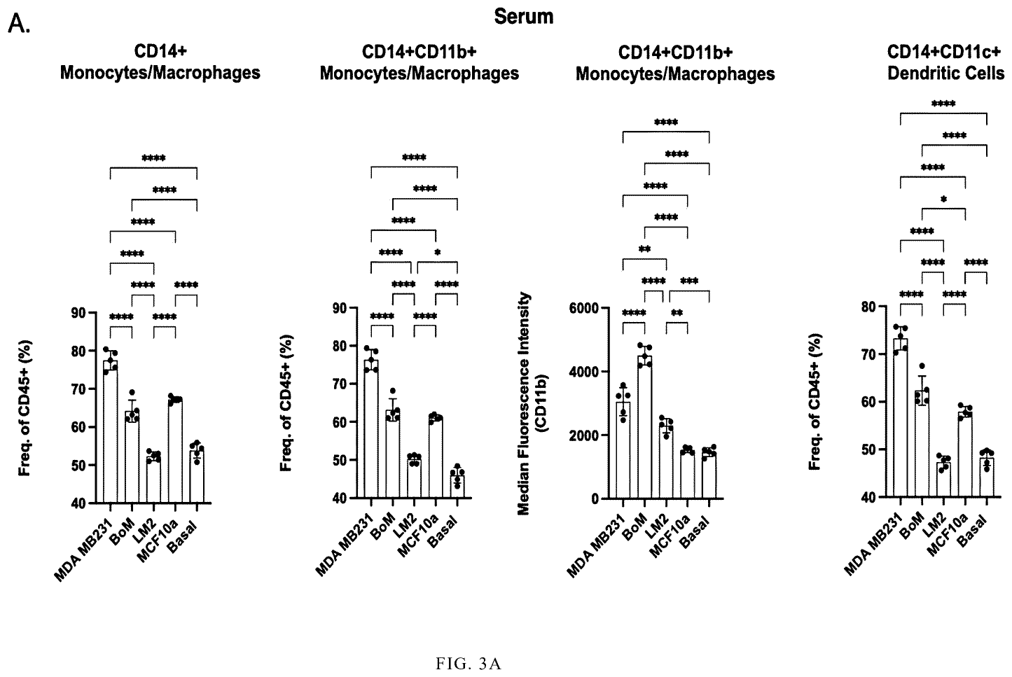

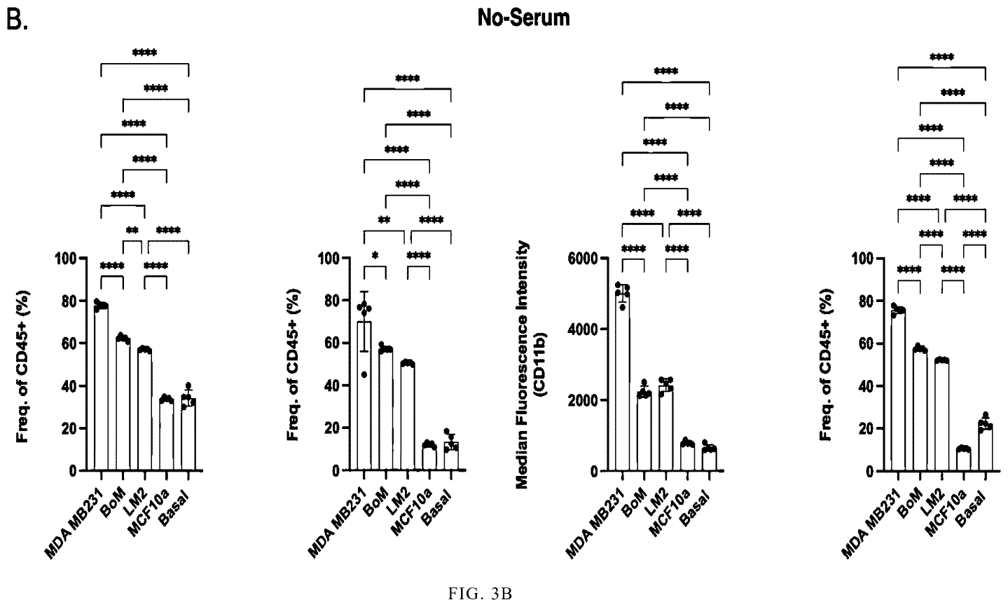

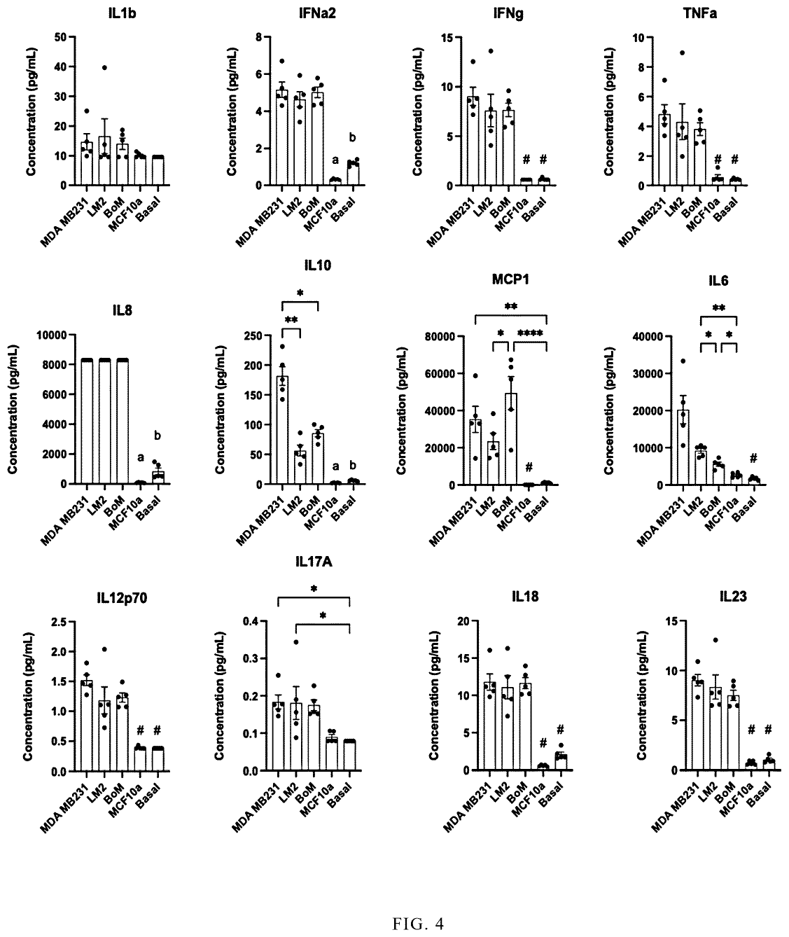

After 7 days using flow cytometry, we observed significant increases in CD45+CD11b+CD14+ monocytes in response to cancer cell conditioned media relative the basal controls, with an example plot shown in FIG. 2B. In conditioned media with serum-treated HSPCs, we still saw many significant increases in CD14+ cells, CD11b+CD14+ monocytes, and CD14+CD11c+ dendritic cells, though our MCF10A normal breast epithelial cells also had increases in myeloid progeny, potentially due to the absence of FBS in the culture of MCF10A cells in normal culture (FIG. 3A). In serum-free conditions, however, the significant effect of myeloid skewing was retained in both the basal media and MCF10A conditioned media groups (FIG. 3B). By performing multiplexed cytokine analysis at Day 6, we saw similarly significant increases in inflammatory cytokines IFN-α2, IFN-g, TNF-α, MCP-1, and IL-8, among others. There were also increases in anti-inflammatory markers, including IL-10, indicating a potentially pan-myeloid cell response in cultures with TNBC-conditioned medium (FIG. 4).

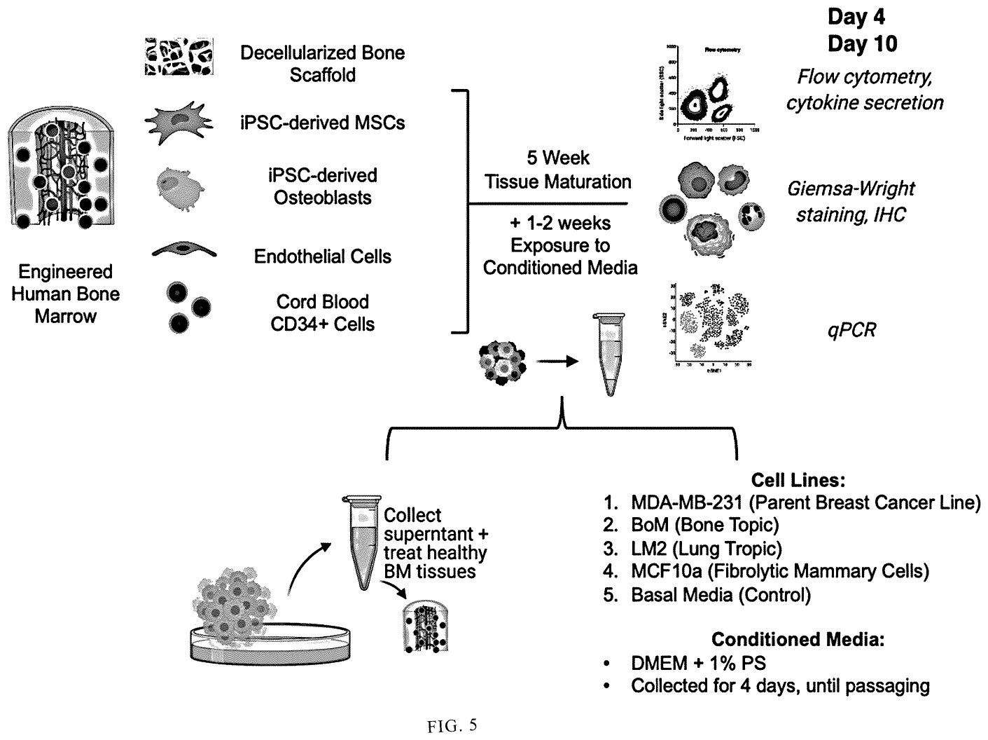

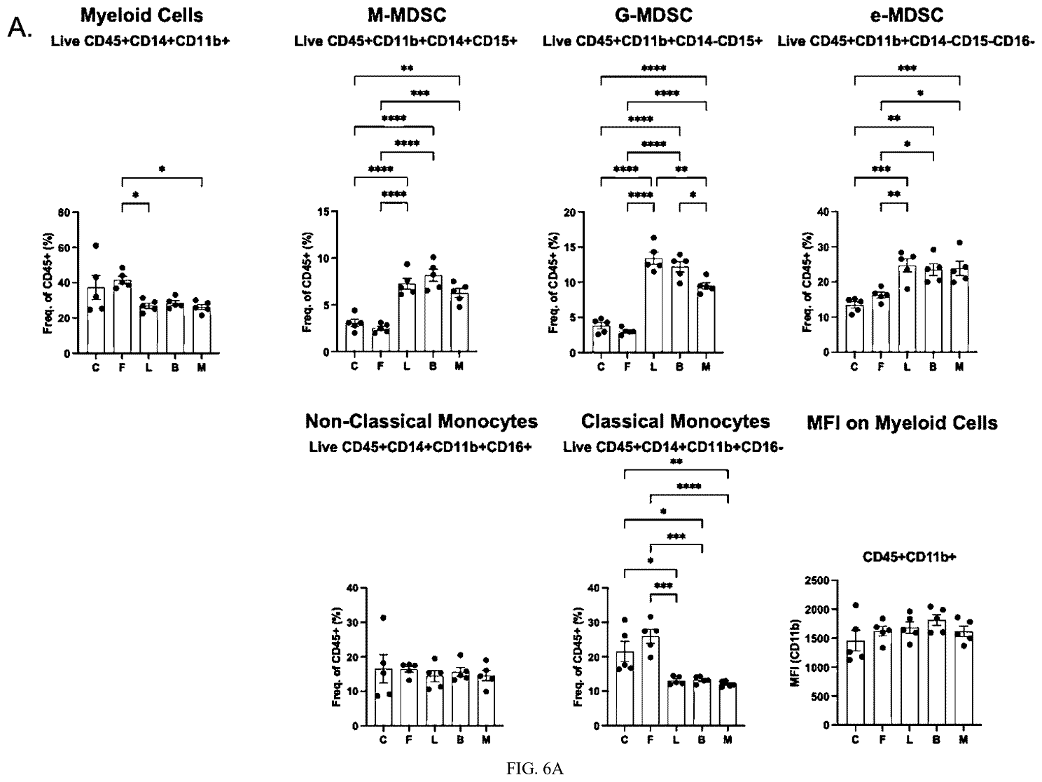

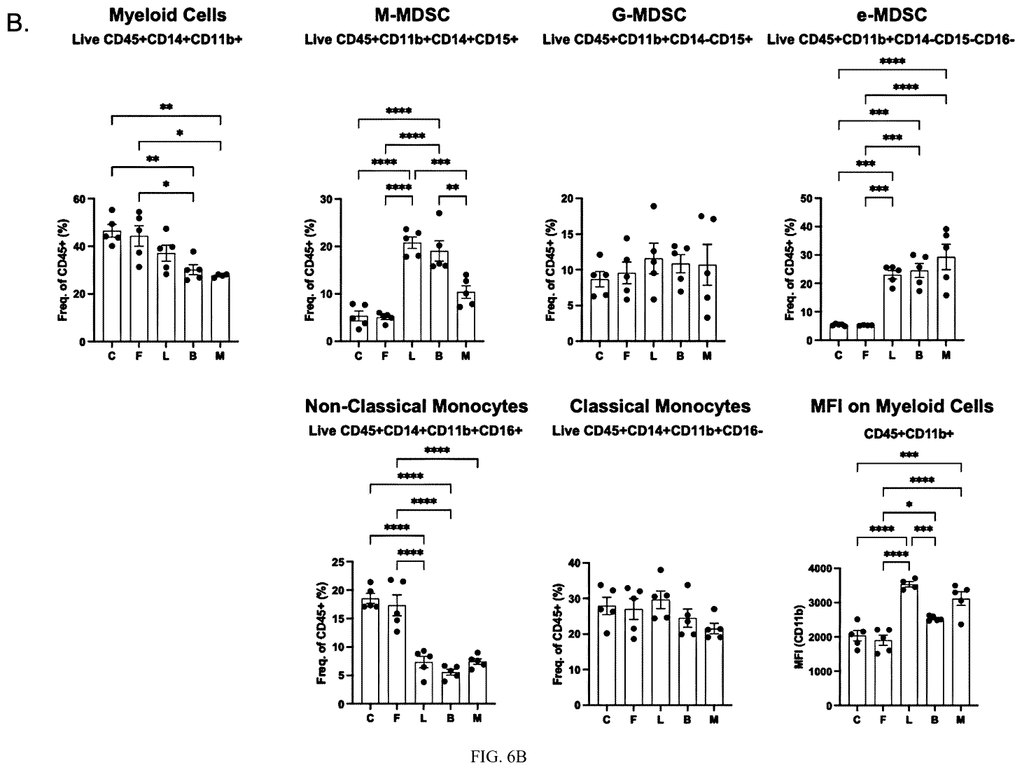

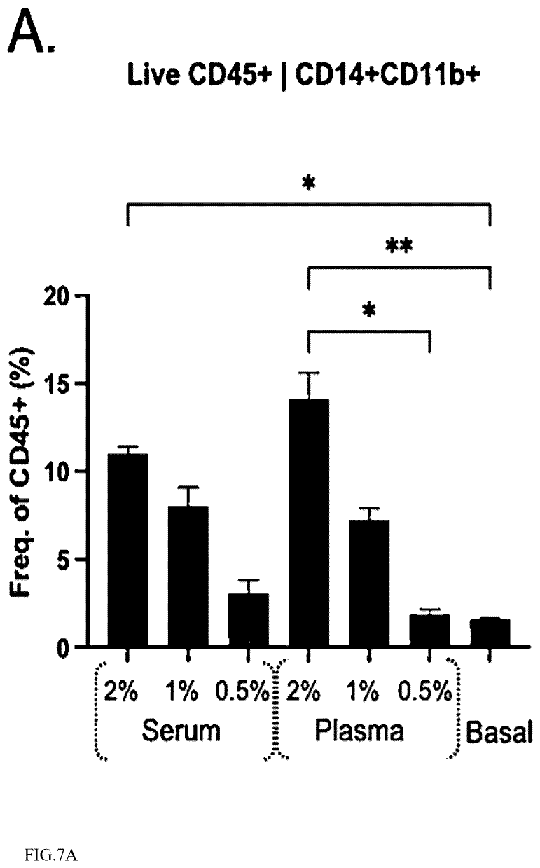

We subsequently confirmed this skewing response in our eBM tissues (FIG. 5). The eBM was cultured for 4-12 days in conditioned media and flow cytometry was performed on the cells in suspension that were released from the tissue. Similar to our previous results, we observed a significant increase in myeloid skewing and identification of monocytic/granulocytic MDSCs that distinguish metastatic culture supernatants from that of non-cancerous epithelial and healthy controls as early as 4 days (FIG. 6A), which were maintained over 10 days in culture (FIG. 6B). At Day 4, cancer conditioned medium caused increases in classical monocytes (CD14+CD16−), versus by Day 10 this increase shifted to an increase in Non-Classical Monocytes (CD14+CD16+). Notably, CD11b+ median fluorescent intensity was significantly increased in all cancer conditioned medium groups at Day 10, indicating that the expression levels of CD11b+ was much higher on CD11b+ myeloid cells than those of the controls (basal control and MCF10A conditioned medium controls). Importantly, all studies were performed using only low exogenous hematopoietic cytokines.

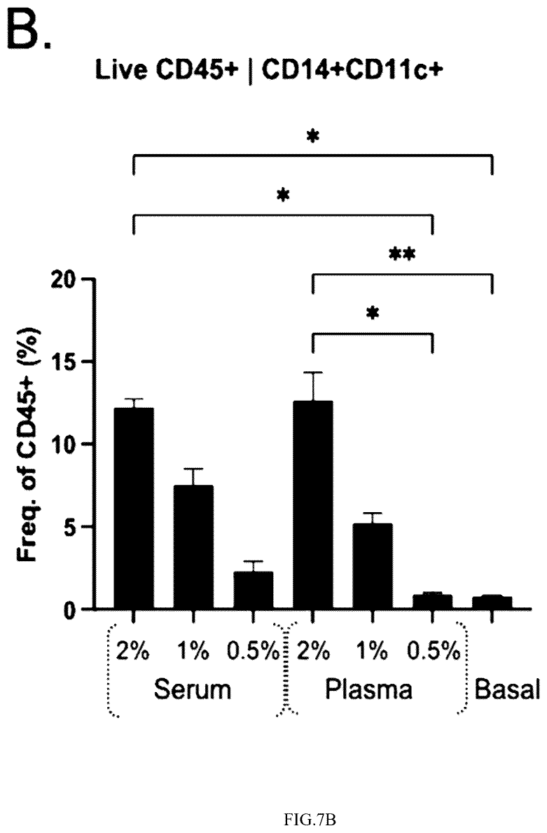

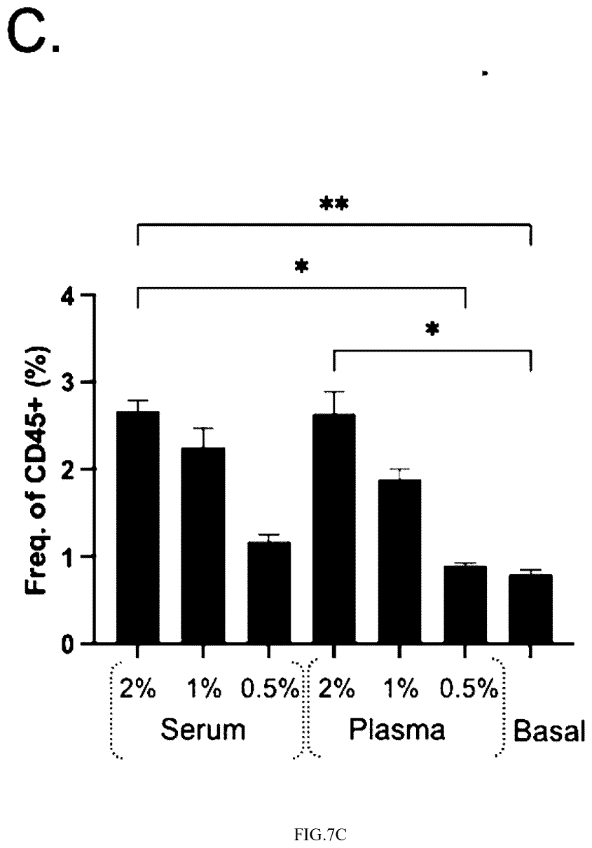

Dosing of patient serum and plasma to understand changes in hematopoietic cells: Primary CB-derived HSPCs were cultured in 2D monolayer culture on ultra-low attachment plates and exposed to either 2%, 1% or 0.5% human TNBC-derived serum or plasma. By looking at the percentages of CD11b+CD14+ monocytes (FIG. 7A), CD14+CD11c+ dendritic cells (FIG. 7B), and CD14+CD15+ MDSCs (FIG. 7C), we confirmed that it was necessary to use either 1 or 2% serum or plasma in our studies, and the concentration of serum/plasma corresponded to increasing myeloid skewing in suspension cultures. In a separate study (not shown), we attempted to use human plasma to visualize these changes in the 3D eBM tissues, though there was a problem with clotting of the plasma with the culture media that prevented further analysis of our tissues for flow cytometry. Healthy and BC serum is preferable for 3D eBM cultures, although accessibility of plasma is easier.

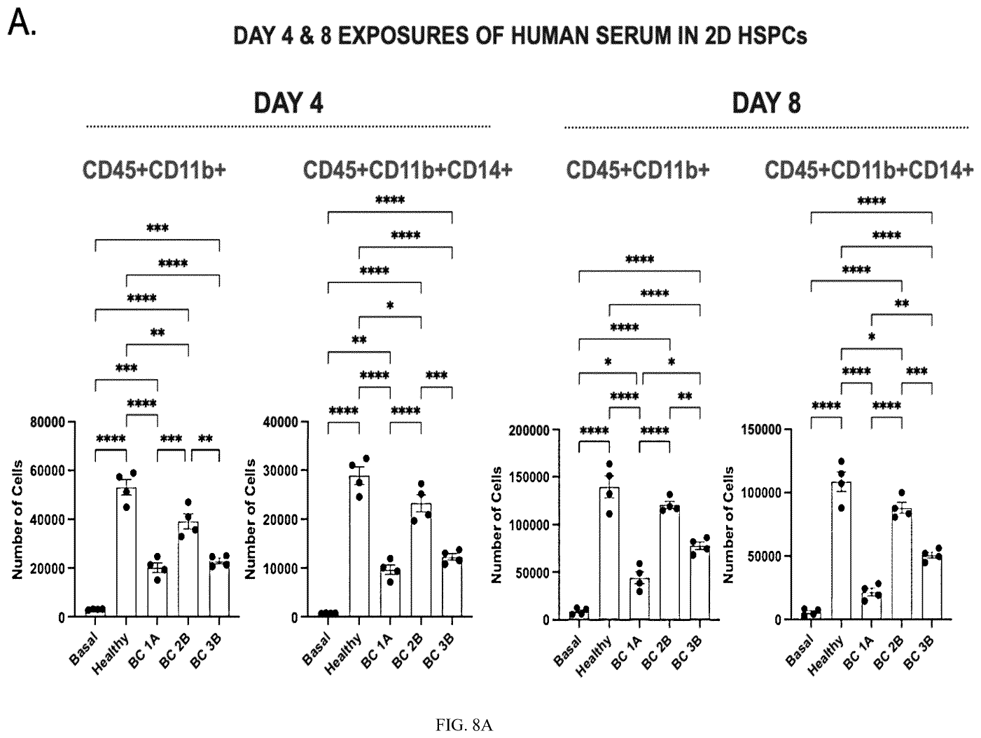

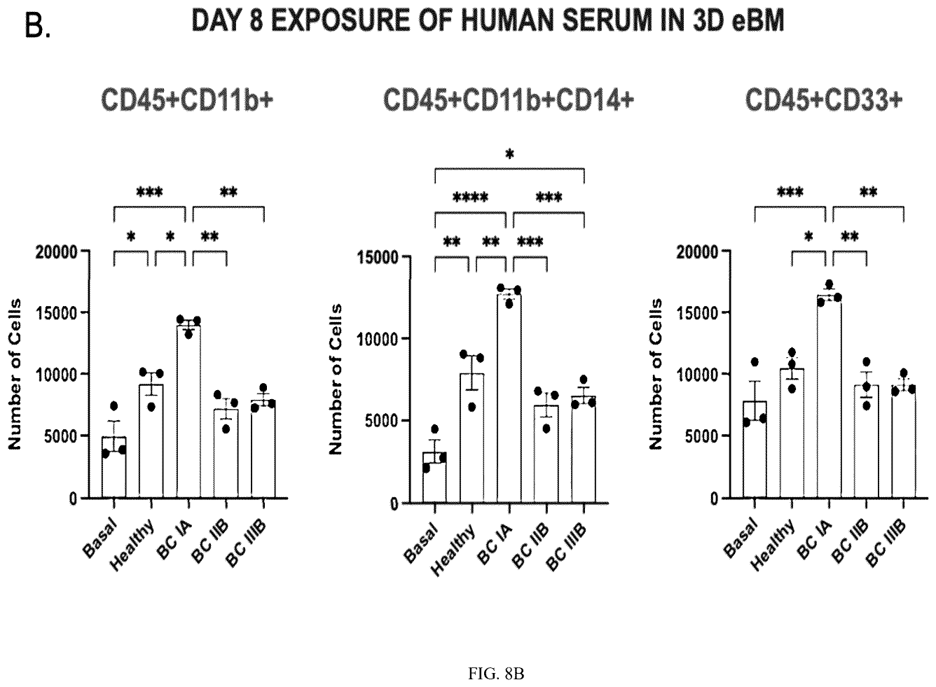

Investigating the effects of patient serum from breast cancer stages to understand hematopoietic and myeloid skewing behavior in eBM microtissues: To understand stage-specific changes to hematopoiesis, we cultured CB-HSPCs in 2D on ultra-low attachment plates and exposed them to serum from healthy and breast cancer patients for a period of 4 to 8 days (FIG. 8A). At Day 4, we immediately saw significant increases in total myeloid cells (CD11b+), as well as CD11b+CD14+CD15+ MDSCs in all stages, as compared to the basal media control (FIG. 8A). Stage 2B had the greatest increase in myeloid cells as compared to the other stages. However, this skewing effect was greatest in the healthy serum, which confounded the results, potentially resulting from little information being provided as to the healthy donor. These effects sustained through re-dosing at Day 4 and through Day 8. In the 3D eBM microtissues, we saw a similar effect at Day 8 in the healthy serum, but the stage that had the greatest increase in significant myeloid cell production was actually at Stage 1A (FIG. 8B). We were also able to consider the expression of CD33+ cells, which its over-expression has been associated with production of M-MDSCs. The human serum purchased was from Discovery Life Sciences, where little information was available regarding BC subtype and treatments of patients. It is preferred to use serum from a vendors with the exact information on stage, subtype, and treatments of patients.

### DISCUSSION

In this work, we describe the use of a human-specific model of the BM to mimic systemic perturbations to hematopoiesis that are caused by cancer metastasis. In both 2D culture of HSPCs and more complex 3D models of hematopoiesis, we demonstrated unique changes to myeloid cell production and differentiation of MDSC populations in response to conditioned medium from metastatic TNBC cell lines, mimicking the systemic cross-talk between pre-metastatic primary tumor cells and the BM, prior to migration of metastatic cells into the secondary metastatic site (i.e., liver, bone, lungs, brain). Further, we established methodologies to show unique skewing of immune populations in the 3D eBM microtissues in response to different types of human serum, derived from either healthy donors or patients with metastatic breast cancer.

Previous work by a number of groups have demonstrated increases in emergency myelopoiesis in metastatic cancer progression. Most interestingly, the expansion of myeloid progenitors prior to metastatic spread was shown by Casbon et al. in breast cancer, demonstrating that granulocyte colony stimulating factor (G-CSF) produced by tumor cells directs the differentiation of immunosuppressive neutrophils responsible for preventing T-cell responses. Myeloid skewing and directed hematopoietic differentiation trajectories have been observed in a number of human conditions, and in many cases of systemic disease, may indicate downstream changes to the blood and immune system in responding to disease. Most recently, Reyes et al. demonstrated increases in suppressive myeloid cells in response to co-culture of HSPCs with plasma from patients with COVID-19 and bacterial sepsis, mimicking the inflammatory responses observed in patients during infection.

We recognize some limitations. First, we chose a number of different conditions in hematopoietic cytokine concentration, media compositions, and cell numbers to optimize the conditions for 2D and 3D HSPC culture. However, we are limited by the number of permutations possible to mimic responses expected in animal models and seen clinically. In addition, access to patient serum was very difficult, especially in cases of treatment-naïve patients at varying stages of breast cancer. We wanted to minimize the variations between donors, especially due to unavoidable variabilities such as age, ethnicity, and time at serum collection. Further, our data only describes a small snapshot of the available changes to hematopoiesis that may be a result of serum concentrations in metastatic progression.

There are overwhelmingly promising benefits to having a reproducible, in vitro model of the human BM in studying hematopoiesis and immune/blood cell production in health and disease. Critically, the study of human malignant hematopoiesis in the context of the human BM is currently limited by culture limitations and models with complex human microenvironmental components. The system here allows for studying personalized medicine approaches to changes in the BM during metastasis, leading to long-term changes to the BM microenvironment that may influence hematopoietic post-remission. The model can also for predicting metastatic responses and early metastatic progression, before the changes to the BM niche have caused downstream metastatic colonization.

Metastasis-induced changes to the BM, in the context of different stages of the metastatic cascade, may differ between different types of breast cancer, including Luminal A, Luminal B, HER2+, and basal-like (triple negative). Each subtype, and within this, each stage (I-IV), can show a unique profile of changes to MDSC emergence; in addition, these changes may lead to further downstream difficulties in preventing reoccurrence of disease. This can be observed in stromal changes in the eBM. The platform may show diagnosing metastatic changes in as short as 1 week.

### Methods

Culture of BC cell lines: Breast cancer cell lines were cultured according to manufacturer's instructions, usually with high glucose DMEM with 1% P/S and 10% FBS. Conditioned media was collected using a basal media for all groups, DMEM and 1% P/S with or without addition of FBS, for 48 hours, sterile-filtered, and stored at −80° C. until use.

Sourcing of breast cancer serum: Serum and plasma was purchased from Discovery Life Sciences, where we were able to purchase serum from different stages but without any indication of treatment type or cancer subtype in breast cancer patients. Serum was thawed at 4° C. and aliquoted for long-term storage at −80° C. without more than two freeze-thaw cycles prior to use.

Culture of 2D HSPCs: CB-HSPCs were expanded with SFEM II basal media (Stem Cell Technologies) with 1% P/S, 1X CD34+ expansion supplement (Stem Cell Technologies), and 1 μM UM729 (Stem Cell Technologies) for 4-5 days prior to use. Cells were then replated onto ultra-low attachment plates and treated with conditioned medium or serum as indicated for 4-8 days prior to flow cytometry analysis.

3D eBM tissues: eBM tissues were formed with seeding of decellularized 8 mm×4 mm×1 mm bone scaffolds. After bone tissue maturation (4-5 weeks of osteogenic media), hematopoietic components were seeded with 11 mg/mL fibrinogen as described previously with 150,000 iMSCs, 150,000 HUVECs, 40,000 CB-HSPCs, with all cells collected and expanded (not direct from thaw) prior to seeding onto tissues. After seeding fibrinogen and cell suspensions, 33 U/mL of thrombin was added to tissues and allowed to polymerize for 25-30 minutes at 37° C. After this, tissues were re-hydrated with SFEM II media supplemented with 1% P/S (Gibco), and 50 ng/mL each of SCF, TPO, and FLT-3L (Peprotech). Until days 3-4, media was supplemented with 33 μg/mL of cell-culture suitable aprotinin (Sigma) and 1 μM UM729 (Stem Cell Technologies) prior to treatment with conditioned medium or serum, at concentrations indicated above. After breast cancer stimuli was applied, cells were collected for flow cytometry.

### FURTHER EXAMPLES

A candidate drug is chosen and administered to the 2D or 3D in vitro model as described herein. The amount and/or identity (e.g., as determined by surface marker expression) of immune skewing associated cells in the 2D or 3D in vitro model is assessed both before and after contacting the 2D or 3D in vitro model with the candidate drug. From the change, or lack thereof, of the amount and/or identity of immune skewing associated cells in the 2D or 3D in vitro model before and after contacting it is thereby determined if the candidate drug reduces or not the amount of immune skewing associated cells in the 2D or 3D in vitro model. A reduction in the amount of immune skewing associated cells in the 2D or 3D in vitro model after contact with the candidate drug indicates that the candidate drug has an ameliorative effect on immune skewing in a bone marrow associated with a metastatic cancer.

The total number of myeloid cells can be identified and/or quantified and, optionally, compared to a predetermined control. The identity and/or quantity of immune skewing associated cells can be assessed by any method known in the art and includes, for example, by flow cytometry. In an example, CD11b+CD14+CD15+ myeloid-derived suppressor cells are identified and/or quantified and, optionally, compared to a predetermined control.

The candidate drug can be a small molecule, a biologic or such.

A serum or plasma sample is obtained from a subject having a tumor, for example a breast cancer tumor, and the metastatic state or metastatic potential of the breast cancer tumor is assessed by contacting a 2D or 3D in vitro model as described herein with conditioned culture media comprising the serum or plasma. The resultant amount and/or identity of immune skewing associated cells in the 2D or 3D in vitro model after contact is determined and correlated with the amount and/or identity of immune skewing associated cells with predetermined control, thus indicating the metastatic state or metastatic potential of the breast cancer tumor in the subject. In an example, the resultant amount and/or identity of immune skewing associated cells in the 2D or 3D in vitro model after contact indicates that metastasis is occurring or will occur. In an example, the total number of myeloid cells is identified and/or quantified. The identity and/or quantity of immune skewing associated cells can be assessed by any method known in the art and includes, for example, by flow cytometry. In an example, CD11b+CD14+CD15+ myeloid-derived suppressor cells are identified and/or quantified and, optionally, compared to a predetermined control.

What is claimed:

1. A 2D or 3D in vitro model of a human bone marrow state comprising human hematopoietic stem and progenitor cells (HSPC) and conditioned culture media comprising (i) media obtained from a primary human triple-negative metastatic breast cancer (TNBC) culture, or (ii) media obtained from a human TNBC cell line, or (iii) plasma or serum obtained from a human subject with TNBC.

2. A 2D or 3D in vitro model of a human bone marrow state comprising human hematopoietic stem and progenitor cells (HSPC) and conditioned culture media comprising (i) media obtained from a culture of human hormone receptor (HR)+ culture or serum, or plasma from a sufferer thereof, (ii) media obtained from a culture of Luminal A, Luminal B, HER2+ or basal-like (triple negative) culture, or serum or plasma from a sufferer thereof.

3. The 2D or 3D in vitro model of claim 1, wherein the HSPC have previously been obtained from a human.

4. The 2D or 3D in vitro model of claim 1, which is a 3D model and which comprises a decellularized bone scaffold.

5. The 2D or 3D in vitro model of claim 1, wherein the TNBC cell line comprises a MDA-MB-231 breast cancer cell line, lung-targeting (LM2), and/or bone-targeting (BOM) cell line.

6. The 2D or 3D in vitro model of claim 1, wherein the conditioned culture media has been supplemented with fetal bovine serum.

7. The 2D or 3D in vitro model of claim 1, wherein the conditioned culture media is not supplemented with fetal bovine serum.

8. The 2D or 3D in vitro model of claim 1, wherein the conditioned culture media comprises plasma or serum obtained from a human subject with TNBC.

9. The 2D or 3D in vitro model of claim 1, wherein the conditioned culture media comprises about 1%-2% plasma or serum obtained from a human subject with TNBC.

10. The 2D or 3D in vitro model of claim 1, wherein the conditioned culture media comprises greater than 0.5% plasma or serum obtained from a human subject with TNBC.

11. The 2D or 3D in vitro model of claim 1, wherein the model comprises a greater number of total myeloid cells relative to an otherwise identical 2D or 3D in vitro model cultured with basal media and no conditioned culture media comprising media obtained from (i), (ii) or (iii).

12. The 2D or 3D in vitro model of claim 1, wherein the model comprises a greater number of CD11b+CD14+CD15+ myeloid-derived suppressor cells (MDSCs) relative to an otherwise identical 2D or 3D in vitro model cultured with basal media and no conditioned culture media comprising media obtained from (i), (ii) or (iii).

13. The 2D or 3D in vitro model of claim 1, wherein the plasma or serum is from a subject having a Stage I, II, III or IV cancer.

14. A method of determining if a candidate drug has an ameliorative effect on immune skewing in a bone marrow associated with a metastatic cancer comprising contacting a 2D or 3D in vitro model of claim 1 with the candidate drug and quantifying the amount of immune skewing associated cells in the 2D or 3D in vitro model before and after contacting with the candidate drug so as to thereby determine if the candidate drug reduces or not the amount of immune skewing associated cells in the 2D or 3D in vitro model, wherein a reduction in the amount of immune skewing associated cells in the 2D or 3D in vitro model after contact with the candidate drug indicates that the candidate drug has an ameliorative effect on immune skewing in a bone marrow associated with a metastatic cancer, and wherein no reduction in the amount of immune skewing associated cells in the 2D or 3D in vitro model after contact with the candidate drug indicates that the candidate drug does not have an ameliorative effect on immune skewing in a bone marrow associated with a metastatic cancer.
